(19) United States
(12) Patent Application Publication
(10) Pub. No.: US 2006/0099171 A1
(43) Pub. Date: May 11, 2006

(54) Mouse glucocorticoid-induced TNF receptor ligand is costimulatory for T cells

(76) Inventors: Masahide Tone, Media, PA (US); Yukiko Tone, Media, PA (US)

Correspondence Address: PEARL COHEN ZEDEK, LLP, 1500 BROADWAY 12TH FLOOR, NEW YORK, NY, 10036, US

(21) Appl. No.: 11/264,029

(22) Filed: Nov. 2, 2005

Related U.S. Application Data
(60) Provisional application No. 60/625,730, filed on Nov. 5, 2004.

(51) Int. Cl. C07H 21/04; C12P 21/02; A61K 39/395; C07K 14/525

(52) U.S. Cl. 424/85.1; 435/69.5; 435/320.1; 435/325; 530/351; 530/350; 536/23.5; 424/145.1

(57) ABSTRACT

The present invention provides mGITRL proteins, nucleotide molecules encoding same, mGITRL messenger RNA molecules, methods of expressing a recombinant gene in an immune cell and of stimulating CD4+CD25− T cells, comprising same or comprising agonist anti-GITR antibodies

### CROSS REFERENCE TO RELATED APPLICATION

This application claims priority of U.S. Provisional Application Ser. No. 60/625,730, filed Nov. 5, 2004, which is hereby incorporated in its entirety by reference herein

### FIELD OF INVENTION

The present invention provides mGITRL proteins, nucleotide molecules encoding same, mGITRL messenger RNA molecules, methods of expressing a recombinant gene in an immune cell and of stimulating CD4+CD25− T cells, comprising same or comprising agonist anti-GITR antibodies.

### BACKGROUND OF THE INVENTION

Mouse glucocorticoid-induced tumor necrosis factor receptor (mGITR) was originally identified in dexamethasone-treated T cell hybridoma cells (Nocentini, G, Giunchi, L et al, (1997) Proc Natl Acad Sci USA 94: 6216-6221) and encodes a 228-aa cysteine-rich protein that is defined as tumor necrosis factor receptor (INFR) superfamily 18 (Tnfrsf 18). The human counterpart and its ligand were characterized soon after (Gumney, A, Marsters, S, et al. (1999) Curr Biol: 9:215-218; Kwon, B, Yu, K et al, (1999) J Biol Chem 274: 6056-6061), the interaction of which activates NF-?B via a INFR-associated factor 2-mediated pathway. The role of mGITR in T cell regulation, particularly regulation of CD4⁺CD25⁻ T cells, has not been well defined.

### SUMMARY OF THE INVENTION

The present invention provides mGITRL proteins, nucleotide molecules encoding same, mGITRL messenger RNA molecules, methods of expressing a recombinant gene in an immune cell and of stimulating CD4+CD25− T cells, comprising same or comprising agonist anti-GITR antibodies.

In one embodiment, the present invention provides a mouse glucocorticoid-induced INF receptor ligand (mGITRL) protein

In another embodiment, the present invention provides a nucleotide molecule encoding a mGITRL protein of the present invention.

In another embodiment, the present invention provides a GITRL messenger RNA molecule having a sequence comprising the sequence set forth in SEQ ID No: 33.

In another embodiment, the present invention provides a GITRL messenger RNA molecule having a sequence comprising the sequence set forth in SEQ ID No: 34.

In another embodiment, the present invention provides an isolated nucleic acid molecule, having a sequence set forth in SEQ ID No: 24.

In another embodiment, the present invention provides a fragment of an isolated nucleic acid molecule of the present invention, wherein the fragment comprises the sequence TATGTTTGGCCTGGTGCCACGATGA (SEQ ID No: 26).

In another embodiment, the present invention provides a fragment of an isolated nucleic acid molecule of the present invention, wherein the fragment comprises the sequence TTGGCCTGGTGCCAC (SEQ ID No: 6).

In another embodiment, the present invention provides a method of expressing a recombinant gene in an immune cell, comprising fusing the gene with a fragment of an isolated nucleic acid molecule, isolated nucleic acid molecule having a sequence set forth in SEQ ID No: 24

In another embodiment, the present invention provides an isolated nucleic acid molecule, having a sequence set forth in SEQ ID No: 26.

In another embodiment, the present invention provides an isolated nucleic acid molecule, having a sequence set forth in SEQ ID No: 6.

In another embodiment, the present invention provides a method of expressing a recombinant gene in an immune cell, comprising fusing the gene with an upstream promoter sequence comprising an isolated nucleic acid molecule of the present invention.

In another embodiment, the present invention provides a method of stimulating a CD4+CD25− T cell, comprising contacting the CD4+CD25− T cell with a glucocorticoid-induced TNF receptor ligand (GITRL) protein.

In another embodiment, the present invention provides a method of stimulating a CD4+CD25− T cell, comprising contacting the CD4+CD25− T cell with an agonist anti-GITR antibody.

### BRIEF DESCRIPTION OF THE FIGURES

FIG. 1: Identification of mGITRL. (A) Amino acid sequences of human (H; SEQ ID No: 22) and mouse (M; SEQ ID No: 23) GITRL were aligned. The predicted transmembrane domain is underlined. (B) Binding of GITR and the putative mGITRL was analyzed by using recombinant GITR-Fc and transfectants expressing this ligand. The putative mGITRL transfectants (filled with gray) and nontransfectants (solid line) were stained with mAb (YGL 386) or recombinant mGITR-Fc (rmGITR-Fc). (C) Binding of mGITR and the mGITRL was analyzed by using mGITR transfectants and recombinant protein of this ligand. mGITR transfectants (filled with gray) and nontransfectants (solid line) were stained with anti-GITR antibody (Anti-GITR Ab) or the recombinant putative mGITRL (rmGITRL). Binding of the recombinant protein was detected with anti-His tag antibody. (D) Signaling through mGITR with the mGITRL was analyzed by a luciferase assay using the NF-?B reporter plasmid. mGITR transfectant (GITR/JE6.1) and nontransfectant (JE6.1) were electropolated with the NF-?B reporter plasmid. Five hours postelectroporation, these cells were harvested and mixed with either growth-arrested (by mitomycin C treatment) HEK293/mGITRL transfectants (GL/293) or HEK293 (293) Mixed combinations are indicated under the graph. Luciferase activities generated by these cells were compared with that in JE6.1 with HEK293.

FIG. 2. Enhancement and inhibition of proliferation of TCR-stimulated T cells with mGITRL. (A) Proliferation assays were performed by using CD4⁺CD25⁻ cells stimulated with mitomycin C-treated, T cell-depleted, female spleen cells and anti-CD3 antibody (Anti-CD3 Ab) or H—Y peptide as antigen (H—Y peptide). CD4+CD25+ cells, recombinant mGITRL (rmGITRL), and/or recombinant hCD40L (rhCD40L) were added (marked +). For suppression assays, CD4⁺CD25⁺ were preactivated. Control cultures in which CD4⁺CD25⁻from CBA/Ca mice were added failed to induce suppression. (B) Proliferation assays using Th1 (R2.2), Th2 (R2.4) clones, and naïve CD4⁺ cells from A1(M)RAG-1−/− mice, with or without recombinant mGITRL. These T cells were stimulated with mitomycin C-treated female spleen cells and different amounts of H—Y peptide as antigen (0-100 nM). (C) Proliferation assays using Th1 (R2.2), Th2 (R2.4) clones, and naïve CD4⁺ cells from A1(M)RAG-1−/− mice with or without mitomycin C-treated mGITRL transfectants (NB2/mGITRL) or its parent cells (NB2) (0-104 cells). These T cells were stimulated with mitomycin C-treated female spleen cells and 10 nM of H—Y peptide as antigen.

FIG. 3. Expression levels of mGITRL mRNA. Expression levels of mGITRL mRNA were analyzed by RT-PCR cDNAs were amplified with mGITRL-specific or HPRT-specific primers. To compare expression levels and minimize PCR artifacts, the number of PCR cycles was kept low, and PCR products were detected by Southern blot hybridization using specific probes (A) cDNAs were prepared by using RNA from indicated organs and cells with an oligo(dT) primer. Where indicated, cells were stimulated with LPS (10 μg/ml) (B) RT-PCR was performed by using RNA from nonstimulated (0 h) and LPS-stimulated (2-24 h) RAW 264 cells or bmDC. LPS stimulation times are indicated above the blot. RI-PCR results were also analyzed by using PhosphorImaging, allowing mRNA levels of mGITRL to be compared with those of HPRT (shown above the blot).

FIG. 4. Cell surface expression of mGITRL. (A) Spleen cells were stained with anti-mGITRL antibody YGL386 (solid line) or an isotype control antibody (dotted line). These cells were co-stained with anti-CD3, anti-B220, or anti-F4/80 antibody and then positive cells were gated. Median fluorescence intensity (MFI) were as follows: B220⁺ B cells: control, 7.7; mGITRL, 14.9; F4/80⁺ macrophages: control, 25.4; mGITRL low, 56 2; mGITRL high, 673.2; and CD3⁺ T cells: control, 11.2; mGITRL, 9.65. (B) Peritoneal cells were also stained with anti-mGITRL antibody YGL386 (solid line) or an isotype control antibody (dotted line). Cells were co-stained with anti-F4/80 antibody, and then positive cells were gated. MFI were: control, 254.8 and MGITRL, 421.7 (C) Nonstimulated (0 h) and LPS-stimulated (6, 12, and 24 h) bmDCs were stained with anti-mGITRL antibody YGL 386 (solid line) or an isotype control antibody (dotted line). bmDC were co-stained with an anti-CD11c antibody (DC maker), and positive cells were gated. MFI values are indicated under the histograms.

FIG. 5. Gene structure and promoter activity of mGITRL. (A) Coding exons are indicated by black boxes, and a 3′ noncoding region is indicated by a gray box. An alternative 3′ noncoding exon is indicated by a white box. Splice joints are indicated by dotted lines. A partial promoter and 5′ noncoding sequence is shown under mGITRL gene structure (SEQ ID No: 24). A major transcription start site (+1), the 3′ end (+52) of the promoter fragments in the luciferase reporter plasmids (in B), and the first ATG are indicated in bold. The TATA box sequence is indicated in bold and underlined. 5′ Ends of the promoter fragments in the luciferase reporter plasmids (in B, D1-D4) are indicated by arrows, and the locations of probes P1, P2, and P3 for EMSA (in FIG. 6A) are indicated by solid lines. (B and C) mGITRL promoter activity was analyzed by luciferase assays. Luciferase activity generated using the reporter plasmids were compared with that generated using the negative control plasmid (no insert) pGL3 -Basic Vector (Basic) in nonstimulated and LPS-stimulated RAW 264 cells. These assays were repeated at least three times (B) The luciferase reporter plasmids were constructed by using the mGITRL promoter fragments. The 5′ end of each promoter fragment is indicated in parentheses. (C) The NF-1 site in the luciferase reporter D6 (in B) was mutated, and the structures of these plasmids used in the luciferase assay are illustrated. The mutated NF-1 site (TTGGCCTGGTGCCAC; SEQ ID No: 6) to TGGCCTGGGAATTC; SEQ ID No: 7) is indicated by an X.

FIG. 6. Binding of transcription factor NF-1 to the mGITRL promoter. (A) The presence of cis-acting elements between −120 and −94 was shown by luciferase assays (FIG. 5B). Oligo probes P1, P2, and P3 for EMSA were designed in this region and its flanking regions, as depicted in FIG. 5A (SEQ ID No: 25-27, respectively). EMSA was performed by using the probes (P1-P3) and nuclear extract from RAW 264 cells. (B) A competition assay was performed by using a 100-fold excess of unlabeled competitor with ³²P-labeled P2 probe. The competitors used are indicated above the gel. Probe P2 sequence and mutated sequences in M1 and M2 are shown under the gel. The transcription factor binding site in P2 is underlined. (C) Super-shift assay was performed by using probe P2 and an anti-NF-1 antibody (marked +). (D) EMSA was performed by using probe P2 and nuclear extracts from un-stimulated (0 h) and LPS-stimulated (2-24 h) RAW 264 cells. NF-1 and probe complexes are indicated. (E) EMSA was performed by using probe P2 and nuclear extracts from nonstimulated (0 h) and LPS-stimulated (2-24 h) bmDC (EMSA). NF-1 in nuclear extracts used for EMSA was detected by immunoblotting using anti-NF-1 antibody (IB).

FIG. 7. Location of hGITRL homologous protein gene on mouse chromosome 1. The amino acid sequence of hGITRL was used to perform a translated BLAST search against the mouse genome database Two homologous peptide sequences (M) were identified and are shown with the AA sequence of the hGITRL (H). Positions of Infsf4 (OX40L) and Infsf6 (FasL) genes are also indicated. Encoding regions of these two homologous peptides were mapped within 9.1 kb. Top and bottom M sequences ale SEQ ID No: 28 and 29, respectively; top and bottom H sequences are SEQ ID No: 30 and 31, respectively.

FIG. 8. Cell surface expression of mGITR and mGITRL on Th1 and Th2 clones. (A) Th1 (R2.2) and Th2 (R2.4) clones were stained with anti-mGITR antibody (solid line) or an isotype control antibody (dotted line). Median fluorescence intensity (MFI) is as follows: Th1 (control, 35.2; mGITR, 504.8) and Th2 (control, 50-5; mGITR, 2641). (B) Th1 (R2.2) and Th2 (R2.4) clones were also stained with anti-mGITRL antibody (solid line) or an isotype control antibody (dotted line). MFI is as follows: Th1 (control, 37.9; mGITRL, 39.2) and Th2 (control, 52.3; mGITRL, 52 3).

FIG. 9. Transcription start sites of mGITRL gene. Total of 215 5′ RACE clones were analyzed to determine the 5′ ends of mGITRL mRNA. mRNA was isolated from nonstimulated (Non), 2-h LPS-stimulated (2 h), and 24-h LPS-stimulated bmDC and nonstimulated RAW264 cells. Analyzed clone number and positions of 5′ ends ale indicated. 42% of all clones contained the same 5′ end, which was defined as position +1. 72% of 5′ ends mapped between −3 and +3, and 11% of 5′ ends mapped between +36 and +40. In 2 h LPS-stimulated bmDC, 15% of 5′ ends mapped between +117 and +120 The DNA sequence of mGITRL gene from −10 to +160 is shown under the RACE results (SEQ ID No: 32). ATG sequences are indicated in italics and bold. Positions of major mRNA start sites are indicated in bold. The coding sequence of the predicted transmembrane region is indicated (TM).

FIG. 10. Structure, alternative splicing, and potential mRNA destabilizing sequences of the MGITRL gene Coding exons and non-coding (5′ and 3′) exons are indicated by black and gray boxes, respectively, and an alternative exon is indicated by a white box. Splice joins are indicated by dotted lines. Partial coding sequence and full 3′ noncoding sequences in exons 3 (SEQ ID No: 34) and 4 (SEQ ID No: 33) are shown under the map. The stop codon and potential poly(A) addition signals ate indicated in bold. A 32-bp sequence (indicated in bold and underlined) of the 3′ noncoding region in the mGITRL isoform mRNA is encoded in exon 3, and the rest of sequence is encoded in exon 4. Potential mRNA destabilizing sequence ATTTA (SEQ ID No: 35) and related sequences are indicated in italics bold.

FIG. 11. Schematic depiction of RACE protocol. In the present invention, dGTP adding a polyG tail, was used instead of dATP adding polyA tail.

### DETAILED DESCRIPTION OF THE INVENTION

The present invention provides mGITRL proteins, nucleotide molecules encoding same, mGITRL messenger RNA molecules, methods of expressing a recombinant gene in an immune cell and of stimulating CD4+CD25− T cells, comprising same or comprising agonist anti-GITR antibodies.

In one embodiment, the present invention provides a mouse glucocorticoid-induced TNF receptor ligand (mGITRL) protein.

In another embodiment, the mGITRL protein has an amino acid (AA) sequence corresponding to SEQ ID No: 23. In another embodiment, the AA sequence is homologous to SEQ ID No: 23. In another embodiment, the AA sequence consists of SEQ ID No: 23. In another embodiment, the AA sequence is a variant of SEQ ID No: 23 Each possibility represents a separate embodiment of the present invention.

In another embodiment, the present invention provides a nucleotide molecule encoding a mGITRL protein of the present invention.

In another embodiment, the nucleotide molecule has a sequence comprising SEQ ID No: 24. In another embodiment, the nucleotide sequence is homologous to SEQ ID No: 24 In another embodiment, the nucleotide sequence consists of SEQ ID No: 24 In another embodiment, the nucleotide sequence is a variant of SEQ ID No: 24. Each possibility represents a separate embodiment of the present invention.

In another embodiment, the present invention provides a GITRL messenger RNA molecule having a sequence comprising the sequence set forth in SEQ ID No: 33.

In another embodiment, the present invention provides a GITRL messenger RNA molecule having a sequence comprising the sequence set forth in SEQ ID No: 34.

In another embodiment, the present invention provides an isolated nucleic acid molecule, having a sequence set forth in SEQ ID No: 24.

In another embodiment, the present invention provides a fragment of an isolated nucleic acid molecule of the present invention, wherein the fragment comprises the sequence TATGTTTGGCCTGGTGCCACGATGA (SEQ ID No: 26).

In another embodiment, the present invention provides a fragment of an isolated nucleic acid molecule of the present invention, wherein the fragment comprises the sequence TTGGCCTGGTGCCAC (SEQ ID No: 6).

In another embodiment, the present invention provides a method of expressing a recombinant gene in an immune cell, comprising fusing the gene with a fragment of an isolated nucleic acid molecule, isolated nucleic acid molecule having a sequence set forth in SEQ ID No: 24. In another embodiment, the fragment is derived from about the N-terminal half of the isolated nucleic acid molecule. In another embodiment, the fragment corresponds to the first 180 nucleotide residues of the nucleic acid molecule. In another embodiment, the fragment is a fragment of the first 180 nucleotide residues of the nucleic acid molecule. Each possibility represents a separate embodiment of the present invention.

In another embodiment, the immune cell wherein the recombinant gene is expressed is a myeloid cell. In another embodiment, the immune cell is a lymphoid cell In another embodiment, the immune cell is a macrophage. In another embodiment, the immune cell is B cell. In another embodiment, the immune cell is a dendritic cell. Each possibility represents a separate embodiment of the present invention.

In another embodiment, the present invention provides an isolated nucleic acid molecule, having a sequence set forth in SEQ ID No: 26. In another embodiment, the isolated nucleic acid molecule has a sequence corresponding to SEQ ID No: 26. In another embodiment, the nucleotide sequence is homologous to SEQ ID No: 26. In another embodiment, the nucleotide sequence consists of SEQ ID No: 26. In another embodiment, the nucleotide sequence is a variant of SEQ ID No: 26. Each possibility represents a separate embodiment of the present invention.

In another embodiment, the present invention provides a fragment of the isolated nucleic acid molecule of claim 14 In another embodiment, the fragment comprises the sequence TTTGGCCTGGTGCCAC (SEQ ID No: 6). Each possibility represents a separate embodiment of the present invention.

In another embodiment, the present invention provides an isolated nucleic acid molecule, having a sequence set forth in SEQ ID No: 6. In another embodiment, the isolated nucleic acid molecule has a sequence corresponding to SEQ ID No: 6. In another embodiment, the nucleotide sequence is homologous to SEQ ID No: 6 In another embodiment, the nucleotide sequence consists of SEQ ID No: 6. In another embodiment, the nucleotide sequence is a valiant of SEQ ID No: 6. Each possibility represents a separate embodiment of the present invention.

In another embodiment, the present invention provides a method of expressing a recombinant gene in an immune cell, comprising fusing the gene with an upstream promoter sequence comprising an isolated nucleic acid molecule of the present invention.

In another embodiment, the present invention provides a method of stimulating a CD4+CD25− T cell, comprising contacting the CD4+CD25− T cell with a glucocorticoid-induced TNF receptor ligand (GITRL) protein.

In another embodiment, the present invention provides a method of stimulating a CD4+CD25− T cell, comprising contacting the CD4+CD25− T cell with an agonist anti-GITR antibody.

The terms “homology,” “homologous,” etc, when in reference to any protein or peptide, refer. In another embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence, similarly indicates a percentage of nucleotides in a candidate sequence that are identical with the nucleotides of a corresponding native nucleic acid sequence.

Homology is, In another embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPI and TREMBL packages.

In another embodiment, “homology” refers to identity to a GITRL sequence (e.g a nucleotide sequence, amino acid sequence, upstream promoter/regulatoly sequence, or mRNA sequence) of the present invention of greater than 70%. In another embodiment, “homology” refers to identity to a GITRL sequence of the present invention of greater than 72%. In another embodiment, “homology” refers to identity to one of SEQ ID No: 1-20 of greater than 75%. In another embodiment, “homology” refers to identity to a GITRL sequence of the present invention of greater than 78%. In another embodiment, “homology” refers to identity to one of SEQ ID No: 1-20 of greater than 80%. In another embodiment, “homology” refers to identity to one of SEQ ID No: 1-20 of greater than 82%. In another embodiment, “homology” refers to identity to a GITRL sequence of the present invention of greater than 83%. In another embodiment, “homology” refers to identity to one of SEQ ID No: 1-20 of greater than 85%. In another embodiment, “homology” refers to identity to one of SEQ ID No: 1-20 of greater than 87%. In another embodiment, “homology” refers to identity to a GITRL sequence of the present invention of greater than 88%. In another embodiment, “homology” refers to identity to one of SEQ ID No: 1-20 of greater than 90%. In another embodiment, “homology” refers to identity to one of SEQ ID No: 1-20 of greater than 92%. In another embodiment, “homology” refers to identity to a GITRL sequence of the present invention of greater than 93% In another embodiment, “homology” refers to identity to one of SEQ ID No: 1-20 of greater than 95%., In another embodiment, “homology” refers to identity to a GITRL sequence of the present invention of greater than 96%. In another embodiment, “homology” refers to identity to one of SEQ ID No: 1-20 of greater than 97%. In another embodiment, “homology” refers to identity to one of SEQ ID No: 1-20 of greater than 98%. In another embodiment, “homology” refers to identity to one of SEQ ID No: 1-20 of greater than 99%. In another embodiment, “homology” refers to identity to one of SEQ ID No: 1-20 of 100%. Each possibility represents a separate embodiment of the present invention.

In another embodiment, homology is determined is via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5× SSC (150 mMNaCl, 15 mM trisodium citiate), 50 mM sodium phosphate (pH 7.6), 5× Denhardt's solution, 10% dextuan sulfate, and 20 μg/ml denatured, sheared salmon sperm DNA.

In one embodiment of the present invention, “nucleic acids” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a mRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miiRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353 -57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G C. Fareed. Each nucleic acid derivative represents a separate embodiment of the present invention.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

In one embodiment, the phrase “contacting a cell” or “contacting a population” refers to a method of exposure, which may be direct or indirect. In one method such contact comprises direct injection of the cell through any means well known in the art, such as microinjection. In another embodiment, supply to the cell is indirect, such as via provision in a culture medium that surrounds the cell, or administration to a subject, or via any route known in the art. In another embodiment, the term “contacting” means that the compound of the present invention is introduced into a subject receiving treatment, and the compound is allowed to come in contact with a receptor in vivo. Each possibility represents a separate embodiment of the present invention.

As provided herein mGITRL and its gene have been identified mGITRL is, in one embodiment, costimulatory for both naïve and primed T cells. Signaling through GITR on CD4⁺CD25⁺ neutralizes the suppressive activity of these cells. This is not mediated by ligand-induced cell death, as shown by analysis of mixed cultures of CD3-activated CD4⁺CD25⁻ (Thy1.1) and CD⁴⁺CD25⁺ cells [hCD52⁺, Thy1.2], with and without recombinant mGITRL, and monitoring death by 7-aminoactinomycin D uptake. No evidence for any increased cell death of CD4⁺CD25⁺ (hCD52⁺, Thy1.2) cells with MGITRL was found. Blockade of the suppressive activity of CD4⁺CD25⁺ is therefore mediated by signaling either via an NF-?B activation pathway of an unidentified signaling mechanism through the GITR endodomain. In another embodiment, the amount of NF-1 in nuclei is regulated by LPS stimulation, thus affecting mGITRL expression.

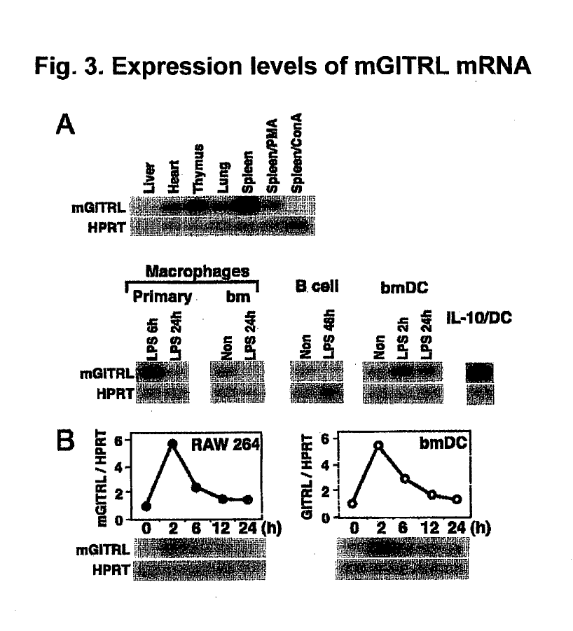

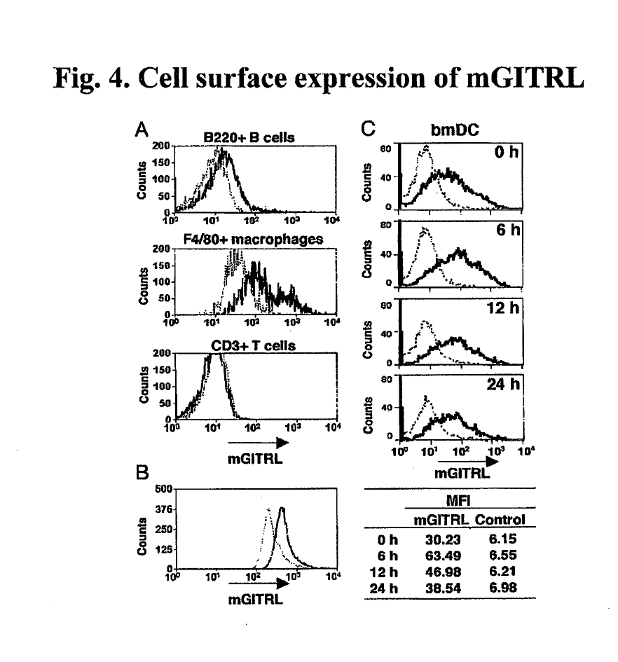

mGITRL expression on the cell surface broadly reflects levels of mGITRL mRNA. However, in another embodiment, mGITRL expression is likely to be controlled in part by posttranslational regulation. The expression level of mGITRL mRNA in bmDC was similar at 0 and 24 h post-LPS stimulation (FIG. 3), but by flow cytometry, the proportion of highly expressing cells was reduced (FIG. 4). In another embodiment, surface mGITRL is regulated by a transport protein.

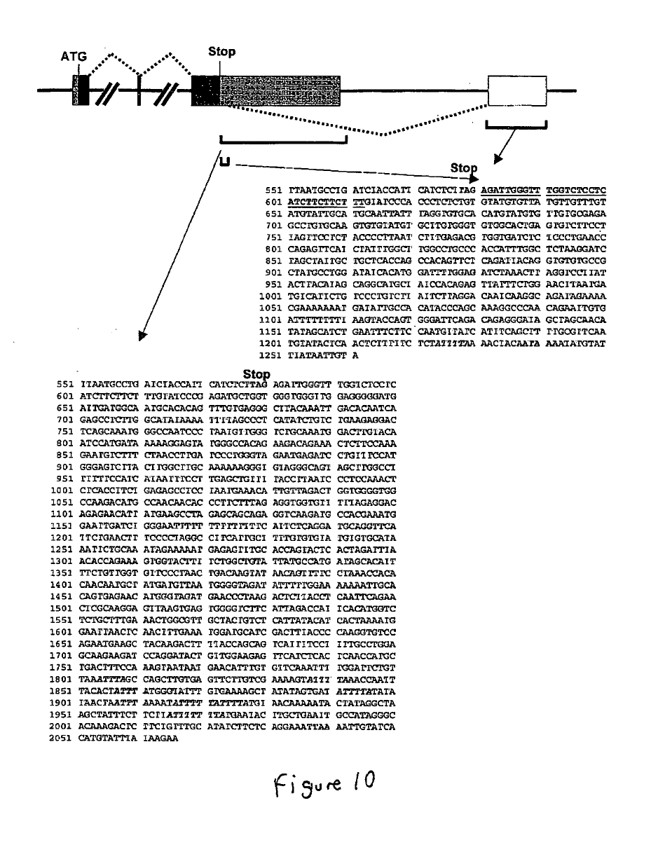

The major mGITRL mRNA species contains a potential RNA destabilization signal AUUUA (SEQ ID No: 4)+AU (SEQ ID No: 5)-rich sequences in the 3′ noncoding region near the 3′ end (FIG. 10). An isoform mRNA, lacking the putative destabilization signal by alternative splicing, was detected in IL-10-treated bmDC that express particularly high levels of mGITRL mRNA (FIG. 3A). Thus, levels of mGITRL mRNA are controlled, in one embodiment, by posttranscriptional regulation.

It is to be understood that any embodiments described herein, regarding peptides, nucleotide molecules, and compositions of this invention can be employed in any of the methods of this invention. Each combination of peptide, nucleotide molecule, or composition with a method represents an embodiment thereof.

In another embodiment, the method entails introduction of the genetic sequence that encodes a protein of this invention. In one embodiment, the method comprises administering to the subject a vector comprising a nucleotide sequence, which encodes a protein of the present invention (Tindle, R. W. et al. Virology (1994) 200:54) In another embodiment, the method comprises administering to the subject naked DNA which encodes a protein of this invention (Nabel, et al PNAS-USA (1990) 90: 11307). Each possibility represents a separate embodiment of the present invention.

Nucleic acids can be administered to a subject via any means as is known in the art, including parenteral or intravenous adminstiation, or in another embodiment, by means of a gene gun. In another embodiment, the nucleic acids are administered in a composition, which correspond, in other embodiments, to any embodiment listed herein.

Vectors for use according to methods of this invention can comprise any vector that facilitates or allows for, the expression of a peptide of this invention. Vectors comprises, in some embodiments, attenuated viruses, such as vaccinia or fowlpox, such as described in, e.g., U.S Pat. No. 4,722,848, incorporated herein by reference. In another embodiment, the vector is BCG (Bacille Calmette Guerin), such as described in Stover et al. (Nature 351:456-460 (1991)). A wide variety of other vectors useful for therapeutic administration or immunization of the proteins of the invention, e.g., Salmonella typhi vectors and the like, will be apparent to those skilled in the art from the description herein

Methods for RACE amplification ale well known in the art, and are described in the Examples. In another embodiment, the following protocol is used for 5′ amplification: One microgram of poly(A)+RNA was reverse transcribed as described above except for the addition of 20?Ci (1 Ci=37 GBq) of[³²P]dCTP and the substitution of 20 pmol of 5RT primer for (dT)₁₇-adaptor . Excess 5RT was removed as follows: the 20-?1 cDNA pool was applied to a Bio-Gel A-5m (Bio-Rad) column (in a 2-ml serological pipette plugged with silane-treated glass wool) equilibrated with 0.05× TE. Void volume (0.8 ml) and 30 one-drop fractions were collected Fractions −4 to +3 relative to the first peak of radioactivity were pooled, concentrated by centrifugation under reduced pressure (Speedvac), and adjusted to 23 ?1. For tailing, 1 ?1 of 6 mM DATP, 6 ?1 of 5× tailing buffer (Bethesda Research Laboratories), and 15 units of terminal deoxynucleotidyl-transferase (Bethesda Research Laboratories) were added, and the mixture was incubated for 10 min at 37° C. and heated for 15 min at 65° C. The reaction mixture was diluted to 500 ? I in TE and 1- to 10-?1 aliquots were used for amplification as described for the 3′-end procedure, except for the substitution of (dT)₁₇-adaptor (10 pmol), adaptor (25 pmol), and amplification (5′ amp, 25 pmol) primers. In another embodiment, the methods described in the product literature for GeneRacer® kit (Invitrogen Life Technologies) are utilized. In another embodiment, any other RACE protocol known in the art is used. Each possibility represents a separate embodiment of the present invention.

Various embodiments of dosage ranges are contemplated by this invention. In one embodiment, the dosage is 20 ?g per peptide per day. In another embodiment, the dosage is 10 ?g mg/peptide/day. In another embodiment, the dosage is 30 ?g mg/peptide/day. In another embodiment, the dosage is 40 ?g mg/peptide/day. In another embodiment, the dosage is 60 ?g mg/peptide/day. In another embodiment, the dosage is 80 ?g mg/peptide/day. In another embodiment, the dosage is 100 ?g mg/peptide/day. In another embodiment, the dosage is 150 ?g mg/peptide/day. In another embodiment, the dosage is 200 ?g mg/peptide/day. In another embodiment, the dosage is 300 ?g mg/peptide/day. In another embodiment, the dosage is 400 ?g mg/peptide/day. In another embodiment, the dosage is 600 ?g mg/peptide/day. In another embodiment, the dosage is 800 ?g mg/peptide/day. In another embodiment, the dosage is 1000 ?g mg/peptide/day. In another embodiment, the dosage is 1500 ?g mg/peptide/day. In another embodiment, the dosage is 2000 ?g mg/peptide/day.

In another embodiment, the dosage is 10 ?g mg/peptide/dose. In another embodiment, the dosage is 30 ?g mg/peptide/dose. In another embodiment, the dosage is 40 ?g mg/peptide/dose. In another embodiment, the dosage is 60 ?g mg/peptide/dose. In another embodiment, the dosage is 80 ?g mg/peptide/dose. In another embodiment, the dosage is 100 ?g mg/peptide/dose. In another embodiment, the dosage is 150 ?g mg/peptide/dose. In another embodiment, the dosage is 200 ?g mg/peptide/dose. In another 15 embodiment, the dosage is 300 ?g mg/peptide/dose. In another embodiment, the dosage is 400 ? g mg/peptide/dose. In another embodiment, the dosage is 600 ?g mg/peptide/dose. In another embodiment, the dosage is 800 ?g mg/peptide/dose. In another embodiment, the dosage is 1000 ?g mg/peptide/dose. In another embodiment, the dosage is 1500 ?g mg/peptide/dose. In another embodiment, the dosage is 2000 ?g mg/peptide/dose.

In another embodiment, the dosage is 10-20 ?g mg/peptide/dose. In another embodiment, the dosage is 20-30 ?g m/peptide/dose. In another embodiment, the dosage is 20-40 ?g mg/peptide/dose. In another embodiment, the dosage is 30-60 ?g mg/peptide/dose. In another embodiment, the dosage is 40-80 ?g mg/peptide/dose. In another embodiment, the dosage is 50-100 ?g mg/peptide/dose. In another embodiment, the dosage is 50-150 ?g mg/peptide/dose. In another embodiment, the dosage is 100-200 ?g mg/peptide/dose. In another embodiment, the dosage is 200-300 ?g mg/peptide/dose. In another embodiment, the dosage is 300-400 ?g mg/peptide/dose. In another embodiment, the dosage is 400-600 ?g mg/peptide/dose. In another embodiment, the dosage is 500-800 ?g mg/peptide/dose. In another embodiment, the dosage is 800-1000 ?g mg/peptide/dose. In another embodiment, the dosage is 1000-1500 ?g mg/peptide/dose. In another embodiment, the dosage is 1500-2000 ?g mg/peptide/dose.

In another embodiment, the total amount of protein per dose or per day is one of the above amounts. In another embodiment, the total protein dose per dose is one of the above amounts.

Each of the above doses represents a separate embodiment of the present invention

### EXPERIMENTAL DETAILS SECTION

### Materials and Experimental Methods

cDNA Cloning and Mapping of cDNA Ends.

The mGITRL gene was identified by using the Celera database. Mapping of 5′ and 3′ ends of the cDNA was performed by RACE as follows:

### Race Protocal

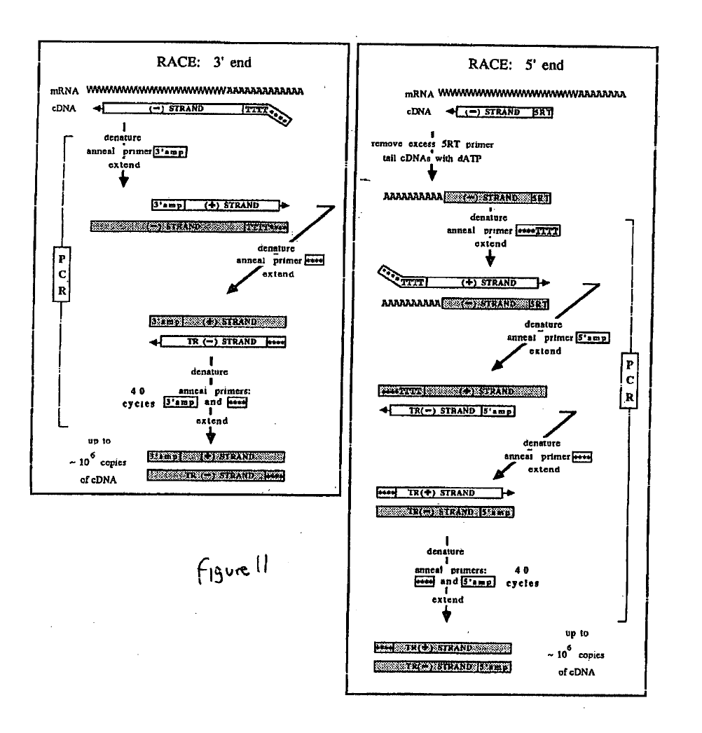

3′-End Amplification of cDNAs (see FIG. 11 for schematic)

Reverse transcription. One microgram of poly(A)⁺ RNA in 16.5 ?1 of water was heated at 65° C. for 3 min, quenched on ice, added to 2 ?1 of 10×RTC buffer (1×RTC buffer is 50 mM Tris-HC, pH 8.15 at 41° C./6 mM MgCl₂/40 mM KC1/1 mM dithiothreitol for each dNTP at 1.5 mM), 0.25 ?1 (10 units) of RNasin (Piomega Biotec, Madison, Wis.), 0.5 ?1 of (dT)₁₇-adaptor (1?g/?1), and 10 units of avian myeloblastosis virus reverse transcriptase (Life Sciences, Saint Petersburg, Fla.), and incubated for 2 hr. at 41° C. The reaction mixture was diluted to 1 ml with TE (10 mM Tris-HCl, pH 7.5/1 mM EDTA) and stored at 4° C.

Amplification. The cDNA pool (1 ?1) and amplification (3′amp) and adaptor primers (25 pmol each) in 50 ?1 of PCR cocktail [10% (vol/vol) dimethyl sulfoxide/1× Taq polymerase buffer (New England Biolabs)/each dNIP at 1.5 mM] were denatured (5 min, 95° C.) and cooled to 72° C. Then 2.5 units of Thermus aquaticus (Taq) DNA polymerase (Perkin-Elmer-Cetus) was added and the mixture was overlaid with 30 ?1 of mineral oil (Sigma) at 72° C. and annealed at 50-58° C. for 2 min. The cDNA was extended at 72° C. for 40 min. Using a DNA Thermal Cycler (Perkin-Elmer-Cetus), 40 cycles of amplification were carried out using a step program (94° C., 40 sec; 50-58° C., 2 min; 72° C., 3 min), followed by a 15-min final extension at 72° C.

5′-End Amplification of cDNAs. (see FIG. 11 for Schematic)

Mix 1?g of total RNA or 0.1?g of poly A+RNA,2?1 of 1 pmol gene specific primer, and DEPC water to the total volume of 12?1 in 0.5 ml PCR tube. Heat the sample at 65° C. for 5 min and place the sample on ice to cool down.

Add 4?1 of 5×RT buffer (with DTT), 2?1 of 5 mMdNTP, 1?1 of RNase Inhibitor and 1?1 of RT, then incubate the sample at 42° C. for 1 hour.

Purify using PCR purification Kit and elute cDNA with 50?1 of elution buffer.

Mix 50?1 of cDNA sample above, 5□1 of 5 mM dGIP (not dATP), 14?1 of tailing buffer and ?1 of TdT. Incubate the sample at 37° C. for 10 minute.

Purify using PCR purification kit and elute with 50?1 of elution buffer.

Then set PCR using gene specific primer and polyC primer.

Southern and RNA Blot Analysis.

Ten-microliter aliquots of RACE reaction products were separated by electrophoresis [1% agarose gel containing ethidium bromide (EtdBr) at 0.5/?g/ml], transferred to GeneScreen (New England Nuclear), and hybridized at high stringency with a ³²P-labeled probe (Bethesda Research Laboratories nick-translation kit), followed by RNA blot analysis.

Cloning and Sequencing of cDNAs.

RACE products were transferred into TE by using spun column chromatography, digested with restriction enzymes that recognize sites in the adaptor or mGITRL sequences and separated by electrophoresis. Regions of the gel containing specific products were isolated, and the DNA was extracted with Glassmilk (Bio 101, San Diego, Calif.) and cloned in a Bluescript vector (Stirtagene, San Diego, Calif.). Plasmids with mGITRL cDNA inserts were identified by colony lift hybridization. Restriction analyses were carried out on plasmid DNA prepared by the alkaline lysis method. Mini-prep plasmid DNA was sequenced with Sequenase (United States Biochemicals, Cleveland).

Specifically for mGITRL, the following primers were used: 5′ RACE primer 1 (TGAGTGAAGTATAGATCAGTG; SEQ ID No: 8), 5′ RACE primer 2 (GCATCAGTAACAGAGCCACTATG; SEQ ID No: 9), 3′ RACE primer 1 (GATGGGAAGCTGAAGATACTG; SEQ ID No: 10), and 3′ RACE primer 2 (GAACTGCATGCTGGAGATAAC; SEQ ID No: 11) For 3′RACE, cDNA was prepared by using a GeneRacer kit (Invitrogen). The mGITRL cDNAs containing the 3′ ends were amplified by using 3′ RACE primers and UAP (Invitrogen).

Cell Culture and Transfection.

The B cell-enriched fraction was prepared from T cell-depleted CBA/Ca mice by passing through splenocytes over a Sephadex G-10 column To prepare bmDC and bm macrophage, bone marrow cells from CBA/Ca mice were cultured for 7 days with granulocyte/macrophage colony-stimulating factor (5 ng/ml). bmDC were separated by gentle aspiration from bm macrophage, which tightly bound to cell culture dishes. If required, these cells were stimulated with LPS (10 μg/ml). To prepare IL-10/DC, 20 ng/ml IL-10 was added to bmDC culture at day 6 and harvested at day 9. Peritoneal cells were isolated from>6 week-old CBA/Ca mice by peritoneal lavage by using ice-cold DMEM containing 0.38% sodium citrate. To isolate primary macrophages, cells were cultured with LPS (10 μg/ml) in bacterial Petri dishes. After 6 or 24 h, binding macrophages on the plastic surface were isolated.

mGITRL transfectants were generated by using NB2 6TG, HEK293 cells, and a mGITRL expression plasmid in pMTF vector. mGITR transfectants were also generated by using a mGITR expression plasmid (in pMTF) and Jurkat (JE6.1) cells. Stable transfectants were selected by G418 (1 mg/ml).

Preparation of Recombinant mGITRL and Anti-mGITRL Antibody.

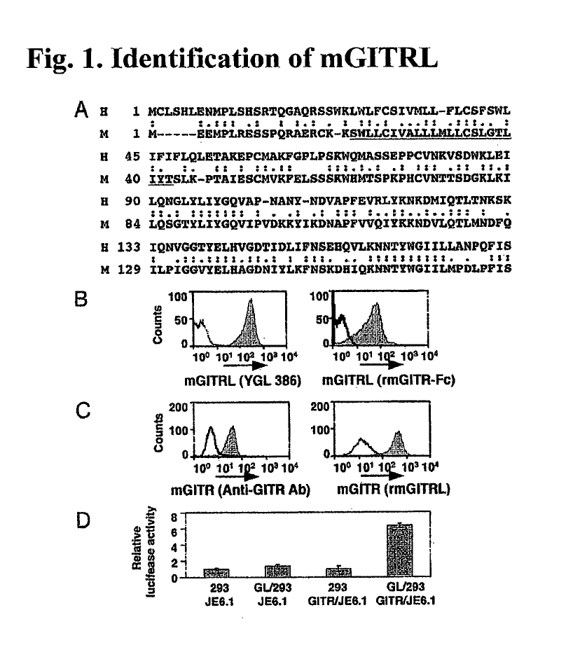

cDNAs encoding extacellular domains of mGITRL (FIG. 1A, amino acid positions 43-173) and human CD40 ligand (hCD40L) (amino acid positions 47-261) were amplified by using PCR primers (mGITRL sense, TCGGATCCTCACTCAAGCCAACTGC: SEQ ID No: 12; mGITRL antisense, AAGAATTCAATCTCTAAGAGATGAATGG: SEQ ID No: 13; hCD40L sense, GTGGGATCCCATAGAAGGTTGGACAAGATAG: SEQ ID No: 14; hCD40L antisense, GTGGAATTCATCAGAGTTTGAGTAAGCCAAAGG: SEQ ID No: 15). Amplified fragments were cloned into BamnHI and EcoRI sites of pRSEI vector (Invitnogen). The resulting plasmids were transferred into Eschetichia coli BL21(DE3)pLysS to produce recombinant proteins by isopropyl-D-thiogalactoside induction. A 6× His tag recombinant protein was purified by using Ni-NTA agaiose (Qiagen, Chatsworth, Calif.), and the eluted protein was dialyzed against PBS.

To produce anti-mGITRL mAb, DA rats were immunized by using this purified recombinant protein. An anti-mGITRL mAB, YGL386 (IgG1), was obtained from the fusion of the immunized rat spleen with a myeloma line, Y3/Ag1.2.3. This antibody was purified by using a protein G column (Amersham Pharmacia Bioscience) and biotinylated. Rat anti-canine CD8 antibody (IgG1) was used as an isotype control antibody.

For flow cytometric analysis using bmDC and transfectants, biotinylated anti-mGITR (R & D Systems, BAF524), anti-mGITRL mAb (YGL386), and recombinant mGITR-Fc (Alexis Biochemicals, Lausen, Switzerland) were used. Allophycocyanin-conjugated streptavidin was used as a secondary reagent. brnDC were costained with FITC-conjugated anti-CD11c antibody (Becton Dickinson Pharmingen). To stain spleen and peritoneal cells, Alexa 488-conjugated YGL386, allophycocyanin-conjugated anti-CD3 (Becton Dickinson), phycoerythrin (PE)-conjugated anti-F4/80 (Becton Dickinson Pharmingen), and PE-conjugated anti-B220 (Becton Dickinson Pharmingen) antibodies were used.

RT-PCR.

To detect mGITRL mRNA, RT-PCR was performed. To compare expression levels and minimize PCR artifacts, the number of PCR cycles was kept low [1.7 cycles for hypoxanthine phosphoribosyltransferase (HPRT), 25 cycles for mGITRL], and PCR products from mGITRL MRNA were detected by Southern blot hybridization using a cDNA probe. Cycle numbers of PCR were determined by preliminary experiments, and under these conditions, PCR was not saturated. The PCR primers used were: mGITRL sense: AGCCTCATGGAGGAAATG (SEQ ID No: 16); mGITRL antisense, ATATGTGCCACTCTGCAGTATC (SEQ ID No 17); HPRT sense, ACAGCCCCAAAATGGTTAAGG (SEQ ID No 18); and HPRT antisense, TCTGGGGACGCAGCAACTGAC (SEQ ID No: 19).

Luciferase Reporter Assay.

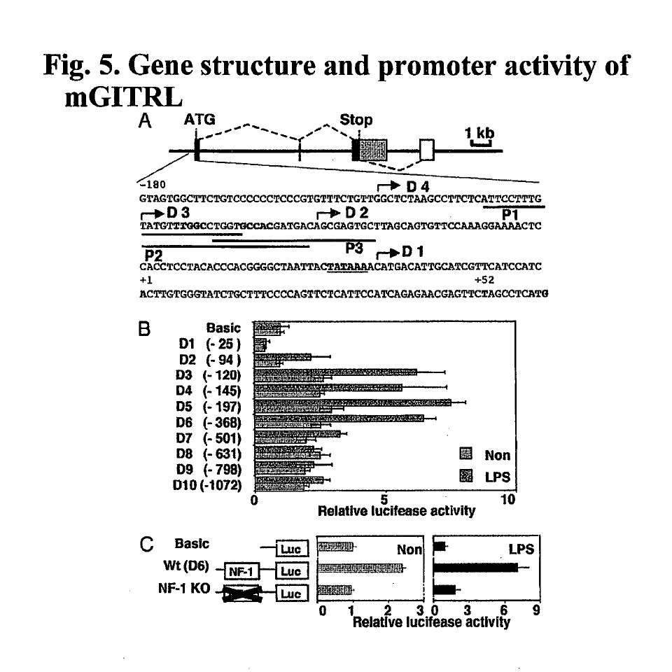

To examine NF-?B activity in mGITR transfectants, a luciferase assay was performed as described in Results pNF-?B luc (Stratagene) was used as a NF-?B reporter plasmid. Ten mGITRL promoter fragments (5′ ends are indicated in FIG. 5B) were cloned into the pGL3-basic Vector (Promega) RAW 264 cells (1.5×10⁷ cells) were transfected with the resulting luciferase reporter plasmids (10 μg) by Gene Pulser (BioRad). If required, cells were stimulated with LPS (10 μg/ml) 5 h postelectroporation. After 48 h culture, cells were harvested, and promoter activities were analyzed by using the Dual-Luciferase Reporter Assay System (Promega). These assays were repeated at least three times, and firefly luciferase activities (mGITRL promoter activities) were normalized to Renilla luciferase (internal control) activities.

Preparation of Nuclear Extracts, Electrophoretic Mobility-Shift Assay (EMSA), and Immunoblotting.

For EMSA, 5 micrograms (?g) of nuclear extract was used. For the competition assay, a 100-fold excess of unlabeled competitor was added to EMSA reaction mixture. To perform the super-shift assay, nuclear extracts in EMSA reaction buffer was incubated with anti-NF-1 antibody (Santa Cruz Biotechnology, H-300) for 15 min, after which probes were added. 10 ?g nuclear extracts and anti-NF-1 antibody (Santa Cruz Biotechnology, H-300) were used for immunoblotting.

Proliferation Assays.

Th1 (R2.2) and Th2 (R2.4) clones, established from the spleen of a female A1(M)RAG-1−/− mouse (6) were used 14 days after antigen stimulation. Naïve CD4+ cells were purified from the spleens of female A1(M)RAG-1−/− mice using the CD4 isolation kit (Miltenyi Biotech, Auburn, Calif.). Total CD4+ T cells were purified from naïve female CBA/Ca mice. CD4+CD25− and CD4+CD25+ cells were separated by cell sorter (MoFlo, Dako Cytomation, Glostrup, Denmark) using FITC-conjugated anti-CD4 and phycoerythrin-conjugated anti-CD25 (both Becton Dickinson Pharmingen) antibodies. Where appropriate, cells were activated by the H—Y peptide (REEALHQFRSGRKPI (SEQ ID No: 20); 1-100 nM), plate-bound or soluble anti-CD3 antibody (145-2C11), and soluble anti-CD28 antibody (37.51).

For proliferation assays, 1×10⁴ clones (R2.2 and R2.4) or 5×10⁴ naïve CD4+ cells from A1(M)RAG-1−/− mice were used. These cells were cultured with 1×10⁵ mitomycin C-treated, T cell-depleted female spleen cells and H—Y peptide (0-100 nM). Where appropriate, recombinant MGITRL (10 μg/ml), recombinant hCD40L (10 μg/ml), mGITRL/NB2 transfectants, or NB2 6TG cells were added to the culture. For suppression assays of CD4+CD25− by CD4+CD25+, cells were activated by anti-CD3 antibody, 5% final concentration of culture supernatant, and proliferation was measured at 48 h by 3H-thymidine incorporation. For the suppression assay with naïve CD4+ cells from A1(M)RAG-1−/− mice, CD4+CD25+ cells were pre-activated overnight with plate-bound anti-CD3 antibody (145-2C11, 10 μg/ml) and soluble/anti-CD28 antibody (37.51, 1 μg/ml), and then treated with mytomycin C. Cultures were established with equal numbers (5×10⁴) CD4+A1(M)RAG-1−/−, CD4+CD25+, and T cell-depleted, mytomycin C-treated, female CBA spleen. Peptide was added at 1-100 nM. After 72 h culture, 0.5 μCi³H-thymidine was added to all cultures and terminated 18 h later.

### EXAMPLE 1

### Identification of mGITRL and Its Gene

### Results

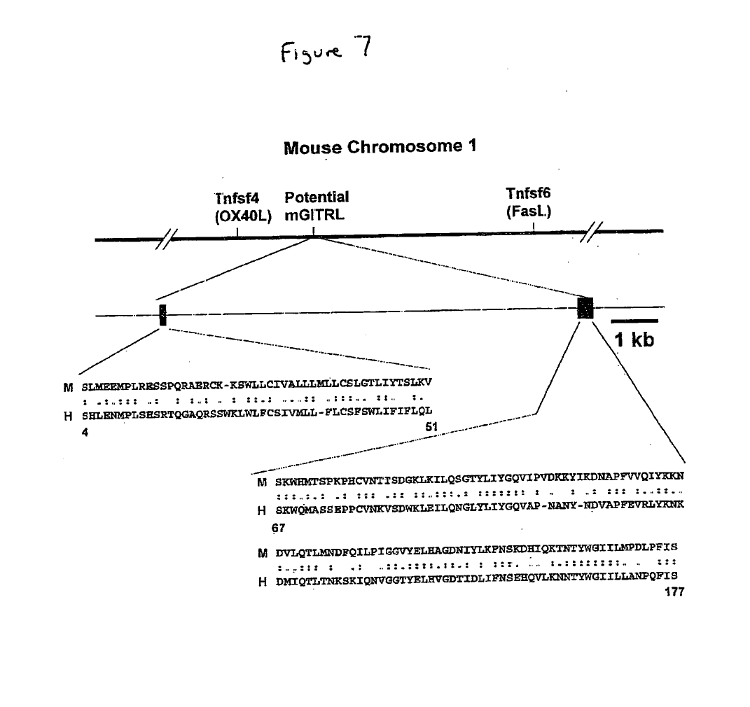

mGITRL was found by searching a mouse genome database by using an amino acid (AA) sequence of human GITR ligand. Two homologous peptide sequences were identified. These two sequences did not overlap, and as encoding regions mapped in the same orientation within 9.1 kb, these two peptides were encoded within one gene (FIG. 7). The gene consisted of at least two exons and mapped to a region between OX40 ligand and Fas ligand genes on chromosome 1, a position equivalent to human GITR ligand gene (chromosome 1q23). RT-PCR and RACE were performed to confirm this finding and determine the full nucleotide sequence of the transcript from the putative gene. Amplified cDNA containing these two exon sequences were obtained from the macrophage cell line RAW 264 and bmDC. This cDNA encoded a 173-AA protein with a type 2 transmembrane topology similar to other TNF family members and having 51% identity with that of human GITR ligand (FIG. 1 A).

To demonstrate that the identified gene product was mGITRL, the ability of the identified gene product to bind to mGITR was examined. A mAb (YGL 386) generated against this gene product was able to bind the surface of transfected mammalian cells expressing the putative mGITRL but not to a control parent cell (FIG. 1B, YGL 386), in a manner similar to a recombinant mGITR Fc immunofusion protein (FIG. 1B, GITR Fc). Furthermore, the recombinant protein from the identified gene was shown to bind an mGITR transfectant but not a control parent cell (FIG. 1C). These results clearly indicate that the identified molecule is mGITRL.

This mGITRL was then shown to be capable of signaling through mGITR. m GITR transfected and control cells were electroporated with a NF-?B/luciferase reporter plasmid. These cells were cultured with either growth-arrested transfectants expressing mGITRL or control parent cells. The luciferase activity (48-h incubation) in GITR-expressing cells cultured with mGITRL-expressing cells was 6-fold greater than the controls (FIG. 1D), showing that NF-?B is activated via signaling through mGITR.

Thus, the mouse glucocorticoid-induced TNF receptor ligand was correctly identified.

### EXAMPLE 2

### Ligand Engagement of mGITR Provides A Costimulatory Signal For T Cell Proliferation

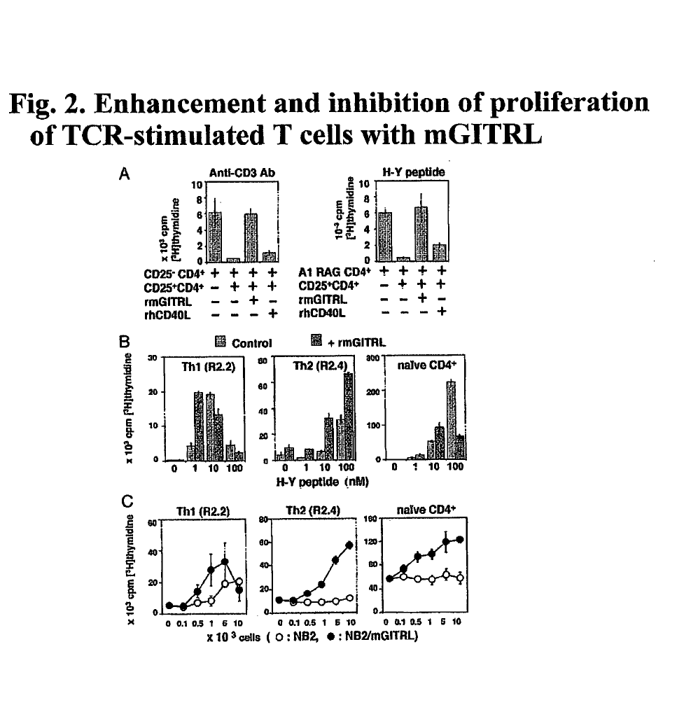

Agonist anti-mGITR antibodies neutralize the suppression of CD3-mediated proliferation of CD4⁺CD25⁻ T cells by CD4⁺CD25⁺ regulatory T cells (4,5). We asked whether the purified mGITRL could do the same. Inhibition of proliferation by CD4⁺CD25⁺ cells was completely neutralized with recombinant mGITRL but not with control, recombinant hCD40L (FIG. 2A, anti-CD3 Ab). We found that mGITRL could also neutralize the suppression of H—Y antigen (male-specific antigen) mono-specific CD4+ T cells from naïve female TCR transgenic mice (FIG. 2A, H—Y peptide).

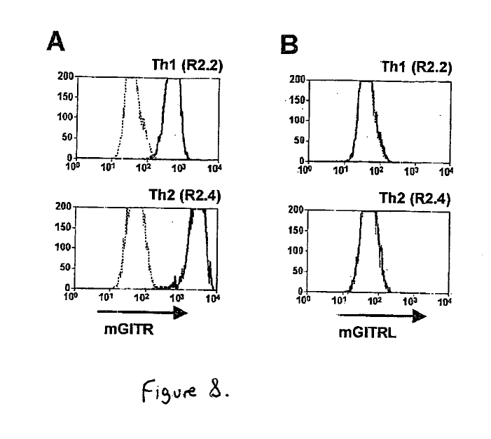

As CD4⁺CD25⁻ T cells and Th cell clones (FIG. 8) also express mGITR to varying extents, the ability of these clones and naïve CD4+ cells from H—Y antigen-specific TCR transgenic mice to respond to signals through mGITR was investigated. The H—Y antigen-specific populations were cultured with antigen-presenting cells, recombinant mGITRL, and varying amounts of antigen (H—Y peptide, 0-100 nM). In the case of the Th1 clone without mGITRL, maximal proliferation was observed with 10 nNM H—Y peptide, with reduced responses at higher peptide doses (FIG. 2B), consistent with IFN- and NO dependent activation-induced apoptosis. mGITRL increased proliferation of Th1 cells with 1 nM peptide but had the opposite effect at the higher 10- and 100 nM concentrations, indicating that GITR signaling had lowered the threshold for activation. The Th2 clone exhibited enhancement of proliferation with mGITRL and H—Y peptide across the range of peptide concentrations tested (FIG. 2B). mGITRL also enhanced proliferation of naïve CD4⁺ cells from the TCR transgenic mice (FIG. 2B) when challenged with 10 nM H—Y peptide, yet just as for the Th1 clone, reduced proliferation with the 100-nM peptide dose.

To test whether mGITR functioned physiologically as a co-stimulatory molecule, the experiments were repeated with a fixed antigen concentration (10 nM) but using mGITRL-transfected cells, to more closely mimic “natural” ligation of GITR (as a trimer on the cell surface). Compared with nontransfected cells, the proliferation of both Th1 and Th2 clones was enhanced in a dose-dependent manner; although the Th1 clone showed evidence of inhibition at the highest dose of tansfectants (FIG. 2C). Proliferation of naive H—Y antigen-specific T cells from the TCR-transgenic mice was also enhanced in a dose-dependent manner (FIG. 2C) These results confirm our findings obtained with recombinant mGITRL (FIG. 2B).

The results of this Example show that the mGITRL engagement of mGITR acts as a costimulatory signal for T cells.

### EXAMPLE 3

### Distribution of mGITRL

In order to determine which cells expressing mGITRL interact with T cells, mGITRL expression was studied by RT-PCR (FIG. 3A). High levels of mGITRL mRNA were detected in spleen, and these levels were reduced after activation with phorbol 12-myristate 13-acetate (PMA) or Con A. Macrophages, B cells, and DC expressed mGITRL mRNA at high levels, which was reduced by LPS stimulation. By contrast, mGITRL mRNA was not expressed in testing and anti-CD3 antibody-activated T cells, specifically in CD4⁺, CD8⁺, Th1 and Th2 clones, regulatory T cell CD4⁺CD25⁺ cells, other regulatory T cell Tr1-like cells, and Tr1-like clones. To further investigate mGITRL regulation by LPS, a 24 h time course of mGITRL mRNA levels was taken of RAW 264 cells (a macrophage cell line) and bone marrow-derived DC (bmDC) after LPS-stimulation. mGITRL mRNA expression was transiently up-regulated, peaking at 2 h after stimulation and then declining.

Cell surface expression of mGITRL was also analyzed by flow cytometry. In splenic populations, mGITRL expression was observed on CD3-B220⁺ B cells and F4/80⁺ macrophages (FIG. 4A) and F4/80⁺ peritoneal macrophages (FIG. 4B), but not splenic T cells (FIG. 4A) or Th1 and Th2 clones (FIG. 8). Cell surface expression of mGITRL corresponds to the levels of mRNA, as demonstrated below for DC. mGITRL was detected on un-stimulated bmDC and increased on upon 6-h stimulation with LPS, with the proportion of highly expressing cells declining after 12 and 24 h of stimulation (FIG. 4C).

Because mGITRL protein is more stable than its mRNA, changes of protein expression levels of mGITRL were less pronounced than that of mGITRL mRNA, but similar outcomes were observed on both mGITRL mRNA and protein expression (FIGS. 3B and 4C).

Thus, mGITRL is expressed on macrophages, B cells, and DC, with levels initially increasing, then decreasing, after stimulation.

### EXAMPLE 4

### Transcription of mGITRL is Regulated by the Transcription FACTOR NF-1

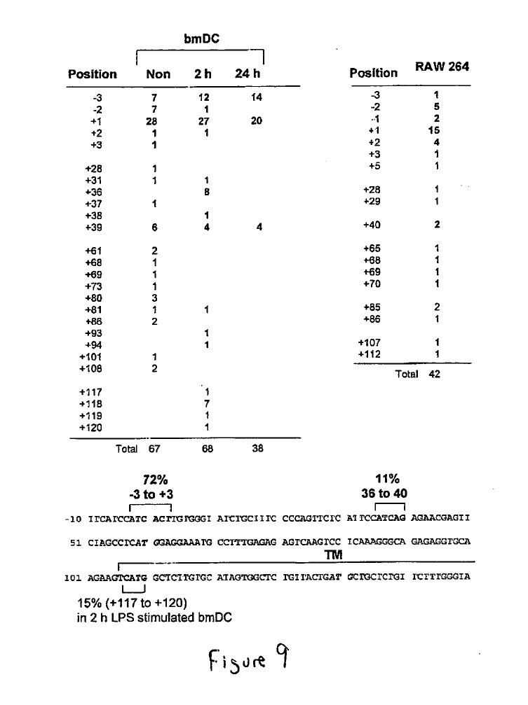

To investigate mGITRL promoter activity, the location of the promoter and gene structure were determined by performing 5′ and 3′ RACE. A total of 215 5′ RACE clones were analyzed (FIG. 9), and the major transcription start site was defined as position +1 (FIG. 5A) Seventy-five percent of 5′ ends were mapped between −3 and +3′. Ten of 13 3′ RACE clones contained 1.46-kb sequences found immediately downstream of the stop codon. In two clones, however, 0.65 kb of the 3′ UTR (0.68 kb) is located 1.9 kb further downstream, indicating that this mRNA is generated by alternative splicing. The gene structure is shown in FIG. 5A and FIG. 10.

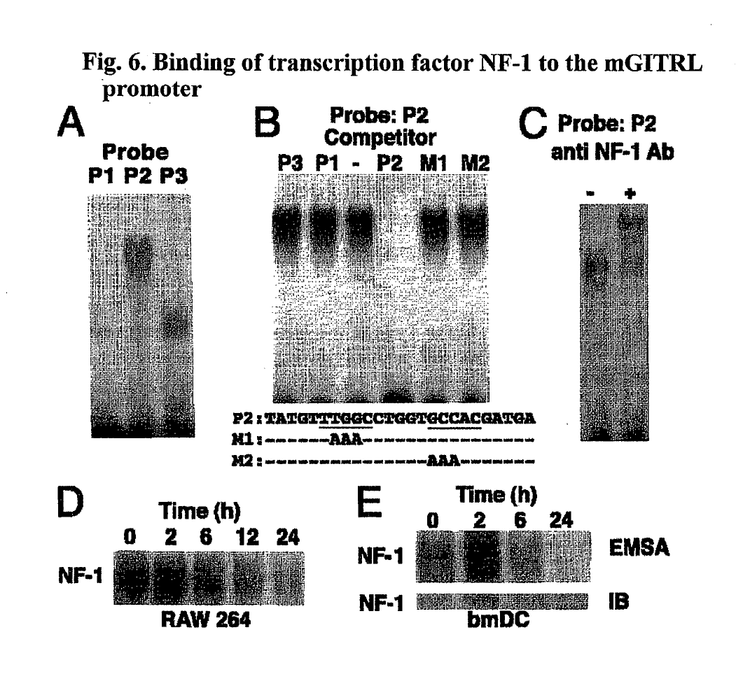

A TATA box sequence was found in a legion 30 bp upstream of a major cluster of transcription start sites (FIG. 5A), indicating that mGITRL gene expression is regulated by a TATA type promoter. A luciferase reporter assay was performed by using deletion mutants of this promoter (FIGS. 5A and B) in RAW 264 cells. Significant reduction of promoter activity was observed by deletion of a 27-bp sequence from −120 (D3) to −94 (D2) in both nonstimulated and LPS-stimulated cells. Transcription factor binding to this region was investigated by an EMSA (FIG. 6) using three probes (P1-P3) (FIG. 5A). A very strong complex formation was detected with ³²P-labeled probe P2 (FIG. 6A) that was inhibited with a 100-fold excess of unlabeled P2, and not with P1 and P3 competitors, or mutant oligodeoxynucleotides M1 and M2 (FIG. 6B), showing that P2 contains a critical sequence similar to NF-1 consensus (TTGGCNNNNNGCCAA; SEQ ID No: 1) . A super-shift assay confirmed that transcription factor NF-1 binds to this promoter (FIG. 6C), and mutation of the NF-1 consensus (TGCCA to GAATT; SEQ ID No: 2 and 3, respectively) resulted in a large reduction of promoter activity (FIG. 5C).

EMSA was also performed by using nuclear extracts from RAW 264 cells and bmDC that were stimulated with LPS for different amounts of time (FIGS. 6D and E, EMSA). The NF-1 complex formation with probe P2 increased in both cell types upon stimulation with LPS for 2 h, then decreased after longer stimulation times. Immuno-blotting with anti-NF antibody IB yielded a similar result to the EMSA (FIG. 6E), indicating that the amount of NF-1 in nuclei is regulated by LPS stimulation.

Thus, NF-1 is a key transcription factor controlling mGITRL gene expression. The upstream sequence TTGGCNNNNNGCCAA (SEQ ID No: 21) plays an important role in mGITRL gene expression.

### EXAMPLE 5

### Model For mGITRL Action

The above results support the following sequence of events for T cell stimulation: (i) In testing APC, constitutive expression of mGITRL MRNA is determined by NF-1, with mGITRL expressed on the cell surface. (ii) Activated APCs initially up-regulate mGITRL to act as a costimulator in T cell interactions. In addition, mGITRL tends to reverse any suppression by CD4⁺CD25⁺ T cells in the local microenvironment. (iii) At later stages of APC activation, mGITRL mRNA and protein are down-modulated. This limits any further costimulatory activity, but releases CD4⁺CD25⁺ regulatory T cells so that they can curtail the ongoing immune response.

What is claimed is:

1. A mouse glucocorticoid-induced TNF receptor ligand (mGITRL) protein.

2. The mGITRL protein of claim 1, wherein said mGITRL protein has an amino acid sequence corresponding to SEQ ID No: 23.

3. A nucleotide molecule encoding the mGITRL protein of claim 1.

4. The nucleotide molecule of claim 3, wherein said nucleotide molecule has a sequence comprising SEQ ID No: 24.

5. A glucocorticoid-induced TNF receptor ligand (GITRL) messenger RNA molecule having a sequence comprising the sequence set forth in SEQ ID No: 33.

6. A glucocorticoid-induced TNF receptor ligand (GITRL) messenger RNA molecule having a sequence comprising the sequence set forth in SEQ ID No: 34.

7. An isolated nucleic acid molecule, having a sequence set forth in SEQ ID No: 24.

8. A fragment of the nucleic acid molecule of claim 7, wherein said fragment comprises the sequence TATGTTTGGCCTGGTGCCACGATGA (SEQ ID No: 26).

9. A fragment of the nucleic acid molecule of claim 7, wherein said fragment comprises the sequence TTGGCCTGGTGCCAC (SEQ ID No: 6).

10. A method of expressing a recombinant gene in an immune cell, comprising fusing said gene with the first 180 nucleotide residues of the nucleic acid molecule of claim 7, or a fragment thereof.

11. The method of claim 10, wherein said immune cell is a myeloid cell.

12. The method of claim 10, wherein said immune cell is a lymphoid cell.

13. The method of claim 10, wherein said immune cell is a macrophage, a B cell, or a dendritic cell.

14. An isolated nucleic acid molecule, having a sequence set forth in SEQ ID No: 26.

15. A fragment of the isolated nucleic acid molecule of claim 14, wherein said fragment comprises the sequence TTGGCCTGGTGCCAC (SEQ ID No: 6).

16. A method of expressing a recombinant gene in an immune cell, comprising fusing said gene with an upstream promoter sequence comprising the isolated nucleic acid molecule of claim 14.

17. An isolated nucleic acid molecule, having a sequence set forth in SEQ ID No: 6.

18. A method of expressing a recombinant gene in an immune cell, comprising fusing said gene with an upstream promoter sequence comprising the isolated nucleic acid molecule of claim 17.

19. A method of stimulating a CD4⁺CD25⁻ T cell, comprising contacting said CD4⁺CD25⁻ T cell with a glucocorticoid-induced TNF receptor ligand (GITRL) protein.

20. A method of stimulating a CD4⁺CD25⁻ T cell, comprising contacting said CD4⁺CD25⁻ T cell with an agonist anti-GITR antibody.


SEQUENCE LISTING (35 sequences)

SEQ ID NO: 1 - DNA, 15 bp, Mus musculus
  misc_feature (6)..(10): n is a, c, g, or t
ttggcnnnnngccaa

SEQ ID NO: 2 - DNA, 5 bp, Mus musculus
tgcca

SEQ ID NO: 3 - DNA, 5 bp, artificial
  mutated sequence
gaatt

SEQ ID NO: 4 - RNA, 5 nt, Mus musculus
auuua

SEQ ID NO: 5 - RNA, 2 nt, Mus musculus
au

SEQ ID NO: 6 - DNA, 15 bp, Mus musculus
ttggcctggtgccac

SEQ ID NO: 7 - DNA, 14 bp, artificial
  mutation of site
tggcctgggaattc

SEQ ID NO: 8 - DNA, 21 bp, Artificial
  primer
tgagtgaagtatagatcagtg

SEQ ID NO: 9 - DNA, 23 bp, Artificial
  primer
gcatcagtaacagagccactatg

SEQ ID NO: 10 - DNA, 21 bp, Artificial
  primer
gatgggaagctgaagatactg

SEQ ID NO: 11 - DNA, 21 bp, Artificial
  primer
gaactgcatgctggagataac

SEQ ID NO: 12 - DNA, 25 bp, Artificial
  primer
tcggatcctcactcaagccaactgc

SEQ ID NO: 13 - DNA, 28 bp, Artificial
  primer
aagaattcaatctctaagagatgaatgg

SEQ ID NO: 14 - DNA, 31 bp, Artificial
  primer
gtgggatcccatagaaggttggacaagatag

SEQ ID NO: 15 - DNA, 33 bp, Artificial
  primer
gtggaattcatcagagtttgagtaagccaaagg

SEQ ID NO: 16 - DNA, 18 bp, Artificial
  primer
agcctcatggaggaaatg

SEQ ID NO: 17 - DNA, 22 bp, Artificial
  primer
atatgtgccactctgcagtatc

SEQ ID NO: 18 - DNA, 21 bp, Artificial
  primer
acagccccaaaatggttaagg

SEQ ID NO: 19 - DNA, 21 bp, Artificial
  primer
tctggggacgcagcaactgac

SEQ ID NO: 20 - protein, 15 aa, Mus musculus
REEALHQFRSGRKPI

SEQ ID NO: 21 - DNA, 15 bp, Mus musculus
  misc_feature (6)..(10): n is a, c, g, or t
ttggcnnnnngccaa

SEQ ID NO: 22 - protein, 177 aa, Homo sapiens
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 23 - protein, 173 aa, Mus musculus
MEEMPLRESSPQRAERCKKSWLLCIVALLLMLLCSLGTLIYTSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKNNTYWGIILMPDLPFIS

SEQ ID NO: 24 - DNA, 241 bp, Mus musculus
gtagtggcttctgtcccccctcccgtgtttctgttggctctaagccttctcattcctttgtatgtttggcctggtgccacgatgacagcgagtgcttagcagtgttccaaaggaaaactccacctcctacacccacggggctaattactataaaacatgacattgcatcgttcatccatcacttgtgggtatctgctttccccagttctcattccatcagagaacgagttctagcctcatg

SEQ ID NO: 25 - DNA, 24 bp, Artificial
  probe
attcctttgtatgtttggcctggt

SEQ ID NO: 26 - DNA, 25 bp, Artificial
  probe
tatgtttggcctggtgccacgatga

SEQ ID NO: 27 - DNA, 24 bp, Artificial
  probe
tggtgccacgatgacagcgagtgc

SEQ ID NO: 28 - protein, 48 aa, Mus musculus
SLMEEMPLRESSPQRAERCKKSWLLCIVALLLMLLCSLGTLIYTSLKV

SEQ ID NO: 29 - protein, 113 aa, Mus musculus
SKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKNNTYWGIILMPDLPFIS

SEQ ID NO: 30 - protein, 48 aa, Homo sapiens
SHLENMPLSHSRTQGAQRSSWKLWLFCSIVMLLFLCSFSWLIFIFLQL

SEQ ID NO: 31 - protein, 111 aa, Homo sapiens
SKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 32 - DNA, 180 bp, Mus musculus
ttcatccatcacttgtgggtatctgctttccccagttctcattccatcagagaacgagttctagcctcatggaggaaatgcctttgagagagtcaagtcctcaaagggcagagaggtgcaagaagtcatggctcttgtgcatagtggctctgttactgatgctgctctgttctttgggta

SEQ ID NO: 33 - DNA, 711 bp, Mus musculus
ttaatgcctgatctaccattcatctcttagagattgggtttggtctcctcatcttcttctttgtatcccaccctctctgtgtatgtgttatgttgtttgtatgtattgcatgcaattatttaggtgtgcacatgtatgtgttgtgcgagagcctgtgcaagtgtgtatgtgcttgtgggtgtgggactgagtgtcttccttagttcctctaccccttaatctttgagacgtggtgatctctccctgaacccagagttcatctatttggcttggcctgcccaccatttggctctaaggatctagctattgctgctcaccagccacagttctcagattacaggtgtgtgccgctatgcctggatatcacatggattttggagatctaaacttaggtccttatacttacatagcaggcatgctatccacagagttatttctggaacttaatgatgtcattctgtccctgtcttattcttaggacaatcaaggcagatagaaaacgaaaaaaatgatattgccacatacccagcaaaggcggaacagaattgtgatttttttttaagtaccagtgggattcagacagagggatagctagcaacatatagcatctgaatttcttccaatgttatcatttcagcttttgcgttcaatgtatactcaactctttttctctattttaaaactacaataaaatatgtatttataattgta

SEQ ID NO: 34 - DNA, 1516 bp, Mus musculus
ttaatgcctgatctaccattcatctcttagagattgggtttggtctcctcatcttcttctttgtatcccgagatgctggtgggtgggttggagggggatgattgatggcaatgcacacagtttgtgagggcttacaaattgacacaatcagagcctcttggcatataaaattttagccctcatatctgtctgaagaggactcagcaaatgggccaatccgtaatgttgggtctgcaaatggacttgtacaatccatgataaaaaggagtatgggccacagaagacagaaactcttccaaagaatgtctttctaaccttgatccctgggtagaatgagatcctgtttccatgggagtcttacttggcttgcaaaaaagggtgtagggcagtagcttggccttttttccatcataatttccttgagctgttttaccttaatccctccaractctcaccttctgagagcctcctaatgaaacattgttagactggtggggtggccaagacatgccaacaacacccttctttagaggtggtgtttttagaggacagagaacattatgaagcctagagcagcagaggtcaagatgccacgaaatggaattgatctgggaattttttttttttttcattctcaggatgcaggttcattctgaactttcccctaggccttcattgcttttgtgtgtatgtgtgcataaattctgcaaatagaaaaatgagagtttgcaccagtactcactagatttaacaccagaaagtggtacttttctggctgtattatgccatgatagcacattttctgttggtgttccgtaagtgacaagtataacagttttcctaaaccacacaacaatgctatgatgttaatggggtagatatttttggaaaaaaattgcacagtgagaacatgggtagatgaaccctaagactcttacctcaattcagaactcgcaaggagttaagtgagtggggtcttcattagaccattcacatggtctctgctttgaaactggcgttgctactgtctcattatacatcactaaaatggaattaactcaactttgaaatggatgcatcgactttaccccaaggtgtccagaatgaagctacaagacttttaccagcagtcattttccttttgcctggagcaagaagatccaggatactgttggaagagttcatctcactcaaccatgctgactttccaaagtaataatgaacatttgtgttcaaattttggattctgttaaatttagccagcttgtgagttcttgtcgaaaagtattttaaaccaatttacactatttatgggtatttgtgaaaagctatatagtgatattttatatataactaatttaaaatatttttattttatgtaacaaaaatactataggctaagctatttcttcttatttttttatgaatacttggtgaattgccatagggcacaaagactcttctgtttgcatatcttctcaggaaattaaaattgtatcacatgtatttataagaa

SEQ ID NO: 35 - DNA, 5 bp, Mus musculus
attta